(19) United States
(12) Patent Application Publication
(10) Pub. No.: US 2026/0001955 A1
(43) Pub. Date: Jan. 1, 2026

(54) Methods of Treating PD-L1 Expressing Cancer

(71) Applicant: Mayo Foundation for Medical Education and Research, Rochester, MN (US)

(72) Inventors: Svetomir N. Markovic, Rochester, MN (US); Wendy K. Nevala, Rochester, MN (US)

(21) Appl. No.: 19/206,661

(22) Filed: May 13, 2025

Related U.S. Application Data
(62) Division of application No. 17/870,726, filed on Jul. 21, 2022, now abandoned.
Application No. 17/870,726 is a division of application No. 16/330,360, filed on Mar. 4, 2019, now Pat. No. 11,427,637.
(60) Provisional application No. 62/383,938, filed on Sep. 6, 2016.

(51) Int. Cl. C07K 16/28; A61K 31/337; A61K 45/06; A61K 47/69; A61P 35/00; C07K 14/76

(52) U.S. Cl. CPC C07K 16/2827; A61K 31/337; A61K 45/06; A61K 47/6929; A61P 35/00; C07K 14/76

(57) ABSTRACT

Described herein are methods, formulations and kits for treating a patient with cancer with nanoparticle complexes comprising a carrier protein, a binding agent and paclitaxel and optionally co-treated with an anti-PD-L1 antibody.

### FIELD OF THE INVENTION

This disclosure relates to novel methods and kits for treating cancer by administering nanoparticle complexes comprising a carrier protein, a binding agent that binds specifically to PD-L1, and paclitaxel. The method may also comprise pretreating a patient suffering from a cancer comprising cancer cells that express PD-L1 with anti-PD-L1 antibody alone, prior to, concurrently with or after administering the nanoparticle complexes.

### STATE OF THE ART

Cancers cells employ a variety of means to escape immune surveillance and thereby continue to proliferate and/or metastasize. For example, many cancer cell types express or overexpress PD-L1 (programmed cell death ligand 1) (B7-H1), the principal ligand of program cell death protein 1 (PD-1). PD-1 is a cell surface receptor on T lymphocytes and is expressed upon activation in mature hematopoietic cells such as T and B cells, NKT cells and monocytes after prolonged antigen exposure (Ishida et al., 1992. EMBO J. 11:3887). Expression of PD-1 and PD-L1 in the tumor microenvironment appears to be a major resistance mechanism to escape immune surveillance. It is hypothesized that PD-L1 binding to PD-1 on T-cells suppresses effector anti-tumor T-cell activity and facilitates immune evasion.

There are several clinical trials that use monoclonal antibodies that either bind to PD-1 or PD-L1 in the treatment of cancers, including lung cancer, bladder cancer, kidney cancer, hematological cancers, breast cancer, colorectal cancer, melanoma and solid cancers. Anti-PD-1 antibodies known in the art include, e.g., Nivolumab (BMS-936558/MDX-1106/ONO-4538; Bristol Myers Squibb), PDR001 (Novartis), and Pembrolizumab (MK-3475) (Merck Sharp & Dohme); anti-PD-L1 antibodies known in the art include, e.g., BMS-936559/MDX-1105 (Bristol Myers Squibb), Atezolizumab (MPDL3280A, Genetech/Roche), MeDI4736 (durvalumab; MedImmune/AstraZeneca), MSB00100718C (avelumab; EMD Serono) (see, e.g., Philips and Atkins “Therapeutic uses of anti-PD-1 and anti-PD-L1 antibodies” International Immunology Vol. 27(1) pp: 39-46).

Despite the antitumor activity of antibodies targeting the PD-1:PD-L1 pathway, resistance to these therapies has been increasingly observed (see, e.g., Lussier et al. J. Immunotherapy of Cancer, 2015, 3:21 and Koyama et al., Nature Communications, 2016 7:1-9 (Published online 17 Feb. 2016)). Thus there remains a need in the art to improve the efficacy of cancer therapeutics.

### SUMMARY

Described herein are methods for treating a patient suffering from a cancer having cancer cells that express a programed cell death ligand 1 (PD-L1), particularly e.g., a PD-L1-expressing cancer cells that have become resistant to immunotherapy with anti-PD-L1 antibodies. The method comprises, or consists essentially of, administering, to a subject in need thereof, e.g., a mammal having cancer cells that express or overexpress PD-L1, a composition comprising a therapeutic amount of nanoparticle complexes comprising (a) a carrier protein, (b) an effective amount of a binding agent having a PD-L1 binding portion that binds to PD-L1 so as to provide directional guidance to the nanoparticle complexes to the cancer cells and (c) an effective amount of paclitaxel.

In one embodiment, the average diameter of the complexes is between 0.1 and 0.9 m. The binding agent may be an anti-PD-L1 antibody, e.g. atezolizumab. The mammal can be a human. The PD-L1-expressing cancer cells may be, e.g., melanoma, renal cell carcinoma, non-small cell lung carcinoma, head and neck squamous cell carcinoma, colorectal cancer, Merkel cell carcinoma, ovarian cancer, bladder cancer and advanced solid tumors.

The carrier protein/paclitaxel/binding agent nanoparticle complexes can be ABRAXANE®/anti-PD-L1 antibody complexes. ABRAXANE® is available from Celgene Corp. and is a nanoparticle formulation that combines paclitaxel with human albumin. The carrier protein (e.g., albumin)/paclitaxel/antibody nanoparticle complexes, or a composition comprising the complexes, can further comprise an alkylating agent. The alkylating agent can be a platinum compound. The platinum compound can be carboplatin. The anti-PD-L1 antibodies can be humanized antibodies. The anti-PD-L1 antibodies can be chimeric antibodies. The composition can be administered by injection.

In one embodiment, the PD-L1-expressing cancer cells are resistant to immunotherapy with anti-PD-L1 antibodies.

In an embodiment of the methods described herein, the complexes are administered in an amount sufficient to deliver a therapeutically effective amount of the paclitaxel.

The anti-PD-L1 antibody suitable for use in the inventions described herein includes Atezolizumab (TECENTRIQ™, Genentech, Inc. A Member of the Roche Group) or a biosimilar version thereof. In some embodiments, the anti-PD-L1 antibody is BMS-936559/MDX-1105 (Bristol Myers Squibb), Atezolizumab (MPDL3280A, Genetech/Roche), MeDI4736 (durvalumab; MedImmune/AstraZeneca), or MSB00100718C (avelumab; EMD Serono).

In an embodiment of the methods described herein, the target of the cancer cells are cells of a solid cancer.

In an embodiment of the methods described herein, the carrier protein/paclitaxel/PD-L1 binding agent nanoparticle complexes, e.g., albumin/paclitaxel/anti-PD-L1 antibody nanoparticle complexes, are lyophilized and may be reconstituted for administration to a subject in need thereof.

An embodiment of the invention includes a method for increasing the duration of uptake of a chemotherapeutic agent by a tumor that expresses PD-L1. The method includes administering the chemotherapeutic agent in a nanoparticle complex comprising a carrier protein, the chemotherapeutic agent (e.g., paclitaxel) and a PD-L1 binding agent, e.g., an antibody that specifically binds to PD-L1, wherein the binding agent provides directional guidance to the nanoparticle complex to the tumor.

As described herein, in vitro mixing of albumin-containing nanoparticles (e.g., ABRAXANE® nanoparticles) and antibodies (e.g., anti-PD-L1 antibodies, such as Atezolizumab) can result in the formation of macromolecular complexes, the characteristics of which (e.g., size, antibody content, or chemotherapeutic drug content) can be customized depending on need. In some cases, such macromolecular complexes can retain antibody mediated target binding specificity, can retain or exhibit enhanced chemotherapeutic tumor cell cytotoxicity, and can exhibit no additional toxicity beyond that of ABRAXANE® nanoparticles alone. As also described herein, contacting ABRAXANE® with an anti-PD-L1 antibody (e.g., Atezolizumab) prior to administration to a human (e.g., a human cancer patient wherein the cancer expresses or overexpresses PD-L1) can result in a complex that, when administered as a complex, has an increased ability to treat a cancer as compared to a treatment regimen that includes administering ABRAXANE® and the anti-PD-L1 antibody separately in a manner that does not form ABRAXANE®/anti-PD-L1 antibody complexes. The methods and materials provided herein can be used to increase the progression-free survival rate in cancer patients. Increasing progression-free survival can allow cancer patients to live longer. Thus the methods and materials provided herein can be used to increase the overall survival rate in cancer patients.

Also described herein are methods for treating a cancer, preferably a cancer comprising cancer cells expressing programed cell death ligand 1 (PD-L1), by administering to a patient an anti-PD-L1 antibody and nanoparticle complexes, which complexes comprise a carrier protein, paclitaxel and a binding agent that specifically binds to PD-L1, e.g., an anti-PD-L1 antibody. As described herein, the treatment of the subject having a cancer, e.g., a cancer that expresses PD-L1, or overexpresses PD-L1, with the PD-L1 antibody in combination with such nanoparticle complexes increases the therapeutic efficacy of the complexes. Preferably, such anti-PD-L1 antibodies are administered prior to treatment with such nanoparticle complexes. Accordingly, an aspect provided herein is a method for treating a patient suffering from a cancer, e.g., a cancer which expresses or overexpresses PD-L1, wherein the patient is treated with a sub-therapeutic amount of an anti-PD-L1 antibody and a therapeutic amount of nanoparticle complexes comprising the carrier, paclitaxel, and anti-PD-L1 antibody. The administration of the sub-therapeutic amount of the anti-PD-L1 antibody is such that it enhances the efficacy of the nanoparticle complexes. Without wishing to be bound by any theory, it is contemplated that administration of a sub-therapeutic amount of the anti-PD-L1 antibody enhances the therapeutic efficacy of the nanoparticle complexes by binding to non-tumor-bound PD-L1 in the body. Treatment with a sub-therapeutic amount of anti-PD-L1 antibody may allow for greater targeting of the nanoparticle complexes to the tumor, decrease the amounts of the carrier protein/paclitaxel/antibody complexes administered to a patient necessary to achieve a desired effect, or both.

In another aspect, provided herein are methods for enhancing the efficacy of carrier protein/paclitaxel/anti-PD-L1 antibody nanoparticle complexes by administering the complexes about 0.5 to 48 hours after pretreatment of a patient with a sub-therapeutic amount of anti-PD-L1 antibody. Preferably, such nanoparticle complexes are administered about 24 hours after the sub-therapeutic amount of anti-PD-L1 antibody.

In another aspect, provided herein are methods for enhancing the therapeutic outcome in a patient suffering from a cancer, e.g., a cancer expressing or overexpressing PD-L1, by treating the patient with a sub-therapeutic amount of an anti-PD-L1 antibody (e.g., an uncomplexed anti-PD-L1 antibody, e.g. not bound to a carrier protein/paclitaxel complex) and co-treating the patients with an effective amount of nanoparticle complexes comprising albumin, paclitaxel, and anti-PD-L1 antibodies wherein the complexes can bind to PD-L1. In one embodiment, the antibodies are arranged on the surface of the complexes.

In another aspect, provided herein are methods for enhancing the therapeutic outcome in a patient suffering from a cancer, e.g., a cancer expressing or overexpressing PD-L1, by treating the patient with a sub-therapeutic amount of the anti-PD-L1 antibody prior to any subsequent treatment with the nanoparticle complexes comprising carrier protein, paclitaxel, and anti-PD-L1 antibodies, wherein the complexes bind to PD-L1. In one embodiment, the antibodies are arranged on the surface of the complexes.

In an embodiment of this invention, the methods described herein are administered to a subject who has a cancer comprising cells that express PD-L1 but which cancer is resistant to immunotherapy with anti-PD-L1 antibodies that are not in complex with nanoparticles comprising a carrier protein-bound chemotherapeutic, e.g., an albumin bound-paclitaxel nanoparticle, e.g., ABRAXANE®.

Examples of cancer cells known to express PD-L1 and thus suitable for treatment with the methods disclosed herein include but are not limited to melanoma, renal cell carcinoma, non-small cell lung carcinoma, head and neck squamous cell carcinoma, colorectal cancer, Merkel cell carcinoma, ovarian cancer, bladder cancer, hematologic cancers, and other solid cancers, which cancers express PD-L1.

In an embodiment, the anti-PD-L1 antibody is atezolizumab or a biosimilar version thereof. Atezolizumab (trade name TECENTRIQ™) is a fully humanized, Fc-modified monoclonal antibody of IgG1 isotype against PD-L1. Other anti-PD-L1 antibodies include MDX-1105, a fully human monoclonal antibody that binds to PD-L1; Avelumab (MSB0010718C, Merck KGaA, Darmstadt, Germany & Pfizer), a fully human monoclonal PD-L1 antibody of isotype IgG1; and Durvalumab (MedImmune/AstraZeneca), an Fc optimized anti-PD-L1 mAb.

In one embodiment, the sub-therapeutic amount of anti-PD-L1 antibody is selected from an amount consisting of about 1%, about 5%, about 10%, about 15%, about 20%, about 25%, about 30%, about 35%, about 40%, about 45%, about 50%, about 55% or about 60% of the therapeutic dosage of anti-PD-L1 antibody. It is contemplated that administration of the sub-therapeutic amount of anti-PD-L1 antibody preferentially blocks circulating PD-L1 with minimal blocking of PD-L1 associated with a tumor. In some embodiments, the sub-therapeutic amount of anti-PD-L1 to be administered to the patient is determined by analyzing the level of circulating PD-L1 in the blood.

In one embodiment, the sub-therapeutic amount of anti-PD-L1 antibody is administered from between about 30 minutes to about 48 hours prior to administration of the albumin/paclitaxel/anti-PD-L1 antibody nanoparticle complexes.

In other aspects provided herein are unit-dose formulations of an anti-PD-L1 antibody, for example, atezolizumab or a biosimilar version thereof, which formulation comprises from about 1% to about 60% of a therapeutic dose of the antibody wherein the formulation is packaged so as to be administered as a unit dose.

In some embodiments, the formulation of anti-PD-L1 antibodies comprises from about 5% to about 20% of a therapeutic dose of atezolizumab or a biosimilar version thereof. The therapeutic dose for atezolizumab, e.g. for locally advanced or metastatic urothelial carcinoma, is recited in the prescribing information. The therapeutic dose is 1200 mg and preferably a subtherapeutic dose ranges from 5% to 20% of the therapeutic dose. In such a preferred embodiment, such a subtherapeutic dose would range from 60 mg to 240 mg, more preferably from 120 mg to 160 mg.

In other aspects, provided herein are kits comprising: (a) an amount of an albumin/paclitaxel/anti-PD-L1 antibody complexes, (b) a unit dose of a sub-therapeutic amount of anti-PD-L1 antibody, and optionally (c) instructions for use.

In one embodiment, the carrier-bound paclitaxel (e.g., albumin-paclitaxel, e.g., ABRAXANE®)/anti-PD-L1 antibody complexes of the kits are lyophilized. The lyophilized complexes may be reconstituted in an aqueous solution prior to administration. The aqueous solution maybe a sterile aqueous solution or the reconstituted aqueous solution may be filtered sterilized through e.g., a 0.2 or 0.22 μm filter.

An embodiment of the invention includes a method for increasing the duration of tumor uptake of a chemotherapeutic agent by administering the chemotherapeutic agent in a nanoparticle complex comprising a carrier protein, paclitaxel, the chemotherapeutic agent and a PD-L1 binding agent, e.g. an anti-PD-L1 antibody, the PD-L1 binding agent providing directional guidance to the nanoparticle complex to the tumor. In some embodiments, the subject receives a subtherapeutic amount of the anti-PD-L1 antibody prior to or concurrently with such nanoparticle complexes.

### BRIEF DESCRIPTION OF THE DRAWINGS

The following figures are representative only of the invention and are not intended as a limitation. For the sake of consistency, nanoparticles using ABRAXANE® and atezolizumab employ the acronym “AA” and the number after AA such as AA130 is meant to confer the average particle size of these nanoparticles (in nanometers, based on Malvern Nanosight analysis).

FIG. 1 shows the binding affinity between atezolizumab and ABX. The Kd was determined to be 1.462×10⁻⁹. Biolayer interferometry (BLItz) (Forte Bioscience) was performed using streptavidin probes.

FIG. 2A shows the particle size distribution for ABX alone (average size of 90 nm) and ABX-atezolizumab nanoparticles (AA; average size of 129 nm), as determined by Mastersizer NS300. FIG. 2B is a photograph of the ABX-atezolizumab nanoparticles from FIG. 8A.

FIGS. 3A-3E show flow cytometry of ABX-atezolizumab nanoparticles (AA130) competing with labeled anti-PD-L1 antibody for binding to a PD-L1 positive human melanoma cell line, C8161. C8161 cells were pre-treated with isotype control antibody (FIG. 3A), no treatment (FIG. 3B), ABRAXANE® (FIG. 3C), atezolizumab (FIG. 3D), or AA130 (FIG. 3E), then labeled with fluorescently-labeled anti-PD-L1 antibody.

FIG. 4 shows the dose-dependent toxicity of ABX (solid line) and AA130 (broken line) on C8161 cells.

FIGS. 5A-5D show the change in tumor volume over time in mice that were injected with 2×10⁶ PD-L1 positive C8161 melanoma tumor cells, then treated by 100 ul IV tail vein injection with saline (FIG. 5A), atezolizumab alone (18 mg/kg; FIG. 5B), ABX alone (45 mg/kg; FIG. 5C) and AA130 (18 mg/kg atezolizumab and 45 mg/kg ABX; FIG. 5D) one time. Tumor growth was monitored 3 times per week. Tumor size was calculated with the equation: (length×width²)/2.

FIG. 6 depicts the survival of the mice from the experiment shown in FIGS. 11A-11D. Kaplan Meier curves were generated using Graph Pad software. The median survival for each group was 14, 13, 16, and 21.5 days for saline, atezolizumab, Abraxane and AA130, respectively. Survival differences between AA130 and all other groups were significant, with p-values of 0.0008 for saline, 0.0015 for atezolizumab, and 0.0113 for ABX.

### DETAILED DESCRIPTION

After reading this description it will become apparent to one skilled in the art how to implement the invention in various alternative embodiments and alternative applications.

However, all the various embodiments of the present invention will not be described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description of the invention is divided into various sections only for the reader's convenience and disclosure found in any section may be combined with that in another section. Titles or subtitles may be used in the specification for the convenience of a reader, which are not intended to influence the scope of the present invention.

### Definitions

Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to a dose amount means that the dose may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

As used herein, the term “sub-therapeutic” is used to describe an amount of antibody that is below the amount of antibody conventionally used to treat a cancer. For example, a sub-therapeutic amount is an amount less than that defined by the manufacturer as being required for therapy.

The term “nanoparticle” “or “nanoparticle composition” as used herein refers to particles having at least one dimension which is less than 5 microns. In preferred embodiments, such as for intravenous administration, the particle is less than 1 micron. For direct administration, e.g., into a tumor, the particle can be larger. Even larger particles are expressly contemplated by the invention.

In a population of particles, the size of individual particles are distributed about a mean. Particle sizes for the population can therefore be represented by an average, and also by percentiles. D50 is the particle size below which 50% of the particles fall. 10% of particles are smaller than the D10 value and 90% of particles are smaller than D90. Where unclear, the “average” size is equivalent to D50. So, for example, AA130 refers to nanoparticles having an average size of 130 nanometers (nm).

The term “nanoparticle” may also encompass discrete multimers of smaller unit nanoparticles. For example, a 320 nm particle comprises a dimer of a unit 160 nm nanoparticle. For 160 nm nanoparticles, multimers would therefore be approximately 320 nm, 480 nm, 640 nm, 800 nm, 960 nm, 1120 nm, and so on as determined by a Mastersizer 2000 (available from Malvern Instruments Ltd, Wocestershire, UK) as described in PCT/US15/54295.

The term “biosimilar” as used herein refers to a biopharmaceutical which is deemed to be comparable in quality, safety, and efficacy to a reference product marketed by an innovator company (Section 351(i) of the Public Health Service Act (42 U.S.C. 262(i)).

The term “carrier protein” as used herein refers to proteins that function to transport antibodies and/or therapeutic agents. The antibodies of the present disclosure can reversibly bind to the carrier proteins. Examples of carrier proteins are discussed in more detail below.

The term “core” as used herein refers to central or inner portion of the nanoparticle complex which may be comprised of a carrier protein, a carrier protein and a therapeutic agent, or other agents or combination of agents. In some embodiments, the antibody may be non-covalently associated (complexed) with the core.

As used herein, the term “enhancing the therapeutic outcome” and the like relative to a cancer patient refers to a slowing or diminution of the growth of cancer cells or a solid tumor, or a reduction in the total number of cancer cells or total tumor burden.

The term “therapeutic agent” as used herein means an agent which is therapeutically useful, e.g., an agent for the treatment, remission or attenuation of a disease state, physiological condition, symptoms, or etiological factors, or for the evaluation or diagnosis thereof. A therapeutic agent may be a chemotherapeutic agent, for example, mitotic inhibitors, topoisomerase inhibitors, steroids, anti-tumor antibiotics, antimetabolites, alkylating agents, enzymes, proteasome inhibitors, or any combination thereof.

As used herein, the term, “binding agent”, “binding agent specific for”, or “binding agent that specifically binds” refers to an agent that binds to a target antigen and does not significantly bind to unrelated compounds. Preferably the binding agent binds to the target antigen with high specificity having a dissociation constant (Kd) of 10⁻⁷ M, 10⁻⁸ M, or 10⁻⁹ M or lower. Preferably the dissociation constant is about 10⁻⁷M to about 10⁻¹⁴ M. Examples of binding agents that can be effectively employed in the disclosed methods include, but are not limited to, lectins, proteins, and antibodies, such as monoclonal antibodies, e.g., humanized monoclonal antibodies, chimeric antibodies, or polyclonal antibodies, or antigen-binding fragments thereof, as well as aptamers, fusion proteins, and aptamers having or fused to an albumin-binding motif. In an embodiment the binding agent is an exogenous antibody. An exogenous antibody is an antibody not naturally produced in a mammal, e.g., in a human, by the mammalian immune system.

As used herein, the term, “anti-PD-L1 binding agent”, “anti-PD-L1 binding agent specific for,” or “anti-PD-L1 binding agent that specifically binds” refers to an agent that binds to a PD-L1 and does not significantly bind to unrelated compounds. Preferably the PD-L1 binding agent binds to PD-L1 with high specificity having a dissociation constant of 10⁻⁷ M, 10⁻⁸ M, or 10⁻⁹ M or lower. Preferably the dissociation constant is about 10⁻⁷ M to about 10⁻¹⁴ M. Examples of anti-PD-L1 binding agents that can be effectively employed in the disclosed methods include, but are not limited to, antibodies, such as monoclonal antibodies, e.g., humanized monoclonal antibodies, chimeric antibodies, or polyclonal antibodies, or antigen-binding fragments thereof, as well as aptamers, fusion proteins, and aptamers. Preferably, the binding agent has or is fused to an albumin-binding motif. In an embodiment the anti-PD-L1 binding agent is an exogenous antibody.

The term “antibody” or “antibodies” as used herein refers to immunoglobulin molecules and immunologically active portions of immunoglobulin molecules (i.e., molecules that contain an antigen binding site that immuno-specifically bind an antigen). The term also refers to antibodies comprised of two immunoglobulin heavy chains and two immunoglobulin light chains as well as a variety of forms including full length antibodies and portions thereof, including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, bifunctional hybrid antibodies (e.g., Lanzavecchia et al., Eur. J. Immunol. 17, 105 (1987)) and single chains (e.g., Huston et al., Proc. Natl. Acad. Sci. U.S.A., 85, 5879-5883 (1988) and Bird et al., Science 242, 423-426 (1988), which are incorporated herein by reference). (See, generally, Hood et al., Immunology, Benjamin, N.Y., 2ND ed. (1984); Harlow and Lane, Antibodies. A Laboratory Manual, Cold Spring Harbor Laboratory (1988); Hunkapiller and Hood, Nature, 323, 15-16 (1986), which are incorporated herein by reference). The antibody may be of any type (e.g., IgG, IgA, IgM, IgE or IgD). Preferably, the antibody is IgG. An antibody may be non-human (e.g., from mouse, goat, or any other animal), fully human, humanized, or chimeric. In an embodiment the antibody is an exogenous antibody. An exogenous antibody is an antibody not naturally produced in a mammal, e.g., in a human, by the mammalian immune system.

The term “dissociation constant,” also referred to as “Kd,” refers to a quantity expressing the extent to which a particular substance separates into individual components (e.g., the protein carrier, antibody, and/or therapeutic agent).

The terms “lyophilized,” “lyophilization” and the like as used herein refer to a process by which the material (e.g., nanoparticles) to be dried is first frozen and then the ice or frozen solvent is removed by sublimation in a vacuum environment. An excipient is optionally included in pre-lyophilized formulations to enhance stability of the lyophilized product upon storage. In some embodiments, the nanoparticle complexes can be formed from lyophilized components (carrier protein, antibody and therapeutic) prior to use as a therapeutic. In other embodiments, the carrier protein, antibody, and therapeutic agent are first combined into nanoparticle complexes and then lyophilized. The lyophilized sample may further contain additional excipients.

The term “buffer” encompasses those agents which maintain the solution pH in an acceptable range prior to lyophilization and may include succinate (sodium or potassium), histidine, phosphate (sodium or potassium), Tris(tris(hydroxymethyl)aminomethane), diethanolamine, citrate (sodium) and the like. In some embodiments, the buffer of this invention has a pH in the range from about 5.5 to about 6.5; and preferably has a pH of about 6.0. Examples of buffers that will control the pH in this range include succinate (such as sodium succinate), gluconate, histidine, citrate and other organic acid buffers.

The term “pharmaceutical formulation” refers to preparations which are in such form as to permit the active ingredients to be effective, and which contains no additional components which are toxic to the subjects to which the formulation would be administered.

“Pharmaceutically acceptable” excipients (vehicles, additives) are those which can reasonably be administered to a subject mammal to provide an effective dose of the active ingredient employed.

The term “reconstitution time” is the time that is required to rehydrate a lyophilized formulation into a solution.

A “stable” formulation is one in which the protein therein essentially retains its physical stability and/or chemical stability and/or biological activity upon storage.

The term “epitope” as used herein refers to the portion of an antigen which is recognized by an antibody. Epitopes include, but are not limited to, a short amino acid sequence or peptide (optionally glycosylated or otherwise modified) enabling a specific interaction with a protein (e.g., an antibody) or ligand. For example, an epitope may be a part of a molecule to which the antigen-binding site of an antibody attaches.

The term “treating” or “treatment” covers the treatment of a disease or disorder (e.g., cancer), in a subject, such as a human, and includes: (i) inhibiting a disease or disorder, i.e., arresting its development; (ii) relieving a disease or disorder, i.e., causing regression of the disease or disorder; (iii) slowing progression of the disease or disorder; and/or (iv) inhibiting, relieving, or slowing progression of one or more symptoms of the disease or disorder. In some embodiments “treating” or “treatment” refers to the killing of cancer cells.

The term “kill” with respect to a cancer treatment is directed to include any type of manipulation that will lead to the death of that cancer cell or at least a portion of a population of cancer cells.

The term “dose” refers to an amount of the antibody or nanoparticle complex given to a patient in need thereof. The attending clinician will select an appropriate dose from a range based, e.g., on the patient's weight, age, health, stage of cancer, level of circulating PD-L1, and other relevant factors, all of which are well within the skill of the art.

The term “unit dose” refers to a dose of the antibody or nanoparticle complex that is given to the patient to provide a desired result. In some instances, the unit dose is sold in a sub-therapeutic formulation (e.g., 10% the therapeutic dose). The unit dose may be administered as a single dose or a series of subdoses. The therapeutic dose for an antibody for a given FDA-approved indication is recited in the prescribing information, for example the therapeutic dose of Atezolizumab, which is approved for the treatment of patients with locally advanced or metastatic urothelial carcinoma, is 1200 mg administered as an intravenous infusion over 60 or 30 minutes every 3 weeks until disease progression or unacceptable toxicity, and preferably a subtherapeutic dose ranges from 5% to 20% of the therapeutic dose. In such a preferred embodiment such a subtherapeutic dose would range from 60 mg/kg to 240 mg/kg, more preferably from 120 mg/kg to 180 mg/kg. The therapeutic dose for an antibody for a given indication where the antibody is not yet FDA approved or the antibody is not yet approved for that indication, will be the amount the correlates to the therapeutic dose that has been approved for other indications, and thus the subtherapeutic dose for the non-FDA approved indications is readily calculated as a percent of the therapeutic dose (e.g., 10% of the therapeutic dose). For example, the therapeutic dose and therefore the subtherapeutic dose of an antibody for the treatment of metastatic melanoma correlates to the therapeutic dose for metastatic cancers in general that has been approved.

Additionally, some terms used in this specification are more specifically defined below.

### Overview

As will be apparent to the skilled artisan upon reading this disclosure, the present disclosure relates to methods for treating a patient having cancer cells that express PD-L1, and particularly cancer cells that are or have become resistant to treatment with anti-PD-L1 antibody immunotherapy, by treating the patient with carrier protein/paclitaxel/anti-PD-L1 antibody nanoparticle complexes containing a therapeutically effective amount of the paclitaxel.

The present disclosure also relates to methods for treating a patient having cancer cells that express PD-L1, and particularly cancer cells that are or have become resistant to treatment with anti-PD-L1 antibody immunotherapy, by treating the patient with a sub-therapeutic amount of an anti-PD-L1 antibody and carrier protein/paclitaxel/anti-PD-L1 antibody nanoparticle complexes containing a therapeutically effective amount of the paclitaxel.

### Anti-PD-L1 Antibodies

In some embodiments, the anti-PD-L1 antibody is atezolizumab or a biosimilar version thereof.

Atezolizumab (TECENTRIQ™, Roche, USA) is a fully humanized, Fc-modified monoclonal antibody of IgG1 isotype against PD-L1. Atezolizumab is a PD-L1 blocking antibody has been approved for the treatment of patients with locally advanced or metastatic urothelial carcinoma (including bladder cancer) and non-small cell lung cancer.

Other anti-PD-L1 antibodies are also known in the art, e.g., BMS-936559/MDX-1105 (Bristol Myers Squibb), MeDI4736 (Durvalumab, MedImmune/AstraZeneca), and MSB00100718C (avelumab, EMD Serono).

In some embodiments, the sub-therapeutic amount of anti-PD-L1 antibody is selected from an amount consisting of about 1%, about 5%, about 10%, about 15%, about 20%, about 25%, about 30%, about 35%, about 40%, about 45%, about 50%, about 55% or about 60% of the therapeutic dosage of anti-PD-L1 antibody.

In some embodiments, the sub-therapeutic amount of anti-PD-L1 antibody is an amount which preferentially blocks circulating PD-L1 without blocking PD-L1 associated with the tumor.

### Complexes

Methods suitable for preparing carrier protein/paclitaxel/anti-PD-L1 antibody, complexes are described, for example, in U.S. Provisional App. No. 62/060,484, filed Oct. 6, 2014; and U.S. Provisional Patent Application Nos. 62/206,770; 62/206,771; and 62/206,772 filed Aug. 18, 2015, as well as PCT Publication Nos. WO2016/057554, filed Oct. 6, 2015; and WO2014/055415, filed Sep. 30, 2013. The contents of each of these applications are specifically incorporated by reference in their entireties. Example 1 below provides one example of a detailed protocol for making such complexes.

The nanoparticle complexes that may be used in the methods described herein may also comprise a PD-L1 binding agent other than an anti-PD-L1 antibody. Such PD-L1 binding agent comprises a PD-L1 binding portion and an albumin-binding motif, wherein the PD-L1 binding agent complexes with a nanoparticle of carrier protein-bound chemotherapeutic, e.g., an albumin bound paclitaxel nanoparticle, e.g., ABRAXANE®, forming a nanoparticle complex that retains the ability to bind to PD-L1, e.g., after lyophilization and reconstitution. For example that PD-L1 binding agent may be a PD-L1-binding aptamer having or fused to an albumin-binding motif, etc.

In some embodiments, the anti-PD-L1 antibody is atezolizumab or a biosimilar version thereof. In some embodiments, the antibodies are a substantially single layer of antibodies on all or part of the surface of the nanoparticle. In some embodiments the nanoparticle complexes comprise between about 100 and 1000 antibodies, or between about 400 and about 800 antibodies.

In some embodiments the carrier protein is albumin, e.g., a human serum albumin. In some embodiments that albumin is a recombinant human serum albumin.

In some embodiments, the complexes further comprise at least one additional chemotherapeutic agent, e.g., a chemotherapeutic agent selected from the group consisting of abiraterone, bendamustine, bortezomib, carboplatin, cabazitaxel, cisplatin, chlorambucil, dasatinib, docetaxel, doxorubicin, epirubicin, erlotinib, etoposide, everolimus, gefitinib, idarubicin, imatinib, hydroxyurea, imatinib, lapatinib, leuprorelin, melphalan, methotrexate, mitoxantrone, nedaplatin, nilotinib, oxaliplatin, pazopanib, pemetrexed, picoplatin, romidepsin, satraplatin, sorafenib, vemurafenib, sunitinib, teniposide, triplatin, vinblastine, vinorelbine, vincristine, and cyclophosphamide.

In some embodiments, the carrier-bound chemotherapeutic is an albumin-bound paclitaxel, e.g., ABRAXANE® (Celgene).

In one embodiment, the antibodies of the nanoparticle complexes are integrated onto and/or into the nanoparticle complexes, e.g. on the surface of an albumin-bound paclitaxel core. In one embodiment, the antibodies of the nanoparticle complexes are arranged on a surface of the carrier protein (e.g., albumin)-bound paclitaxel core. In one embodiment, the antibodies of the nanoparticle complexes are associated with the albumin-bound paclitaxel core. In one embodiment, the antibodies of the nanoparticle complexes are non-covalently associated with (bound to) a carrier protein, e.g. albumin, in the nanoparticle complex. In one embodiment, the carrier protein (e.g., albumin) and paclitaxel are associated (bound to each other) via non-covalent bonds.

In some embodiments of the invention, the composition comprising the nanoparticle complexes may further comprise an additional chemotherapeutic agent. The additional chemotherapeutic agent may be e.g., an alkylating agent, e.g., a platinum compound, e.g., carboplatin.

In some embodiments, the nanoparticle complex sizes are between 0.09 μm to 0.9 μm, between 90 nm and 800 nm, including about 90 nm, 100 nm, 130 nm, 160 nm, 200 nm, 300 nm, 400 nm, 500 nm, 600 nm, 700 nm or 800 nm. In some embodiments, the nanoparticle complex sizes are between about 100 nm and about 225 nm. In other embodiments, the nanoparticle complexes are larger, e.g., from greater than 800 nm to about 3.5 μm. In some embodiments, the particles are multimers of nanoparticle complexes. In some embodiments the nanoparticle complexes have average particle sizes of about 100 nm to about 225 nm, either freshly made or after lyophilization and resuspension in an aqueous solution suitable for injection.

Without being bound by theory, the binding agent is believed to be bound by the carrier protein through hydrophobic interactions, which, by their nature, are weak. Yet the activity of the individual components, as well as their relative relationship in the nanoparticle are preserved despite lyophilization and reconstitution of the composition. It is still further contemplated that binding to the carrier protein, e.g., complexation of the binding agent to the carrier protein, occurs through an albumin binding motif on the binding agent, and/or an antibody-binding motif on the carrier protein. Albumin-binding motifs and antibody-binding motifs are described in PCT Application No. PCT/US17/45643, filed Aug. 4, 2017, which is incorporated herein by reference in its entirety. In some embodiments, the binding agent is a non-therapeutic and non-endogenous human antibody, a fusion protein, e.g., fusion of an antibody Fc domain to a peptide that binds a target antigen, or an aptamer.

### Treatment Methods

In one aspect is provided a method for treating a patient having a cancer which expresses PD-L1, the method comprising administering to the patient a therapeutically effective amount of anti-PD-L1/albumin/paclitaxel nanoparticles to treat the cancer. In one embodiment, the method comprises selecting a patient having a cancer which expresses PD-L1. In one embodiment, the method comprises selecting a patient having a cancer which expresses PD-L1 and is resistant to treatment with a checkpoint inhibitor immunotherapy. In one embodiment, the checkpoint inhibitor immunotherapy comprises anti-PD-L1 antibodies that are not part of a nanoparticle complex as described herein.

In one aspect is provided a method for treating a patient in need thereof, wherein the patient is treated with a sub-therapeutic amount of an anti-PD-L1 antibody and albumin/paclitaxel/anti-PD-L1 antibody nanoparticle complexes containing a therapeutically effective amount of paclitaxel, such that the administration of the sub-therapeutic amount of the anti-PD-L1 antibody enhances the efficacy of the nanoparticle complexes. A subject in need thereof may be a subject afflicted with a cancer wherein the cancer cells express or over express PD-L1. The subject may also be afflicted with a cancer wherein the cancer cells express or over express PD-L1 but are resistant to treatment with an anti-PD-L1 antibody immunotherapy. In one embodiment, the sub-therapeutic amount of anti-PD-L1 antibodies is not in a nanoparticle complex comprising a carrier protein (e.g., albumin) bound chemotherapeutic (“uncomplexed” anti-PD-L1 antibody).

In one embodiment, the method comprises selecting a patient having a cancer wherein the cancer cells express or over express PD-L1. In one embodiment, the method comprises selecting a patient having a cancer wherein the cancer cells express or overexpress PD-L1 but are resistant to treatment with an anti-PD-L1 antibody immunotherapy. Methods are known in the art for determining whether a tumor comprises cancer cells expressing PD-L1, e.g., the Ventana PD-L1 (sp263) Assay (Roche), which was approved by the FDA as a complementary diagnostic to provide PD-L1 status on patients with metastatic urothelial cancer, and the PD-L1 IHC 28-8 pharmDx assay (Dako, Agilent Pathology Solutions).

In some embodiments of this invention, the nanoparticle complexes comprise a PD-L1 binding agent other than an anti-PD-L1 antibody. In some embodiments of this invention, the nanoparticle complexes comprise an anti-PD-L1 antibody that is the same antibody as the “uncomplexed” anti-PD-L1 antibody. In some embodiments of this invention, the nanoparticle complexes comprise an anti-PD-L1 antibody that is a different antibody than the “uncomplexed” anti-PD-L1 antibody.

The patient may be co-treated with a sub-therapeutic amount of an anti-PD-L1 antibody and carrier protein (e.g., albumin)/paclitaxel/anti-PD-L1 antibody nanoparticle complex.

For the sake of clarification, “co-treatment” refers to treatment of the cancer expressing PD-L1 with an anti-PD-L1 antibody prior to, concurrently with, or immediately after administration of the carrier (e.g., albumin)/paclitaxel/anti-PD-L1 antibody nanoparticle complexes, such that the anti-PD-L1 antibody is capable of binding circulating PD-L1.

In one embodiment, the anti-PD-L1 antibody is administered in a sub-therapeutic dose prior to administration of the nanoparticle complexes. In this embodiment, the administration of the anti-PD-L1 antibody occurs about 0.5 hours to about 48 hours prior to administration of the nanoparticle complexes.

In another embodiment, the anti-PD-L1 antibody composition is administered between 0.5 hours prior to and up to 0.5 hours after administration of the nanoparticle complexes. In this embodiment, it is contemplated that such administration will nevertheless result in binding of some of the circulating PD-L1 by the antibody.

In yet another embodiment, the antibody composition can be administered up to 2 hours post administration of the nanoparticle complexes.

In a preferred aspect, there is provided methods for enhancing the efficacy of albumin/paclitaxel/anti-PD-L1 antibody nanoparticle complexes by administering the albumin/paclitaxel/anti-PD-L1 antibody nanoparticle complexes about 0.5 hours to 48 hours after pretreatment of a patient with a sub-therapeutic amount of anti-PD-L1 antibody. Preferably, such nanoparticle complexes are administered about 24 hours after the sub-therapeutic amount of anti-PD-L1 antibody.

In another aspect, there is provided methods for enhancing the therapeutic outcome in a patient suffering from a cancer expressing PD-L1 which patient is selected to be treated with nanoparticles comprising albumin, paclitaxel and anti-PD-L1 antibodies, which method comprises treating the patient with a sub-therapeutic amount of the anti-PD-L1 antibody prior to any subsequent treatment with the nanoparticles.

In another aspect, there is provided methods for enhancing the therapeutic outcome in a patient suffering from a cancer overexpressing PD-L1, the method comprising co-treating the patient with a sub-therapeutic amount of the anti-PD-L1 antibody and with an effective amount of nanoparticle complexes comprising albumin-bound paclitaxel and anti-PD-L1 antibodies.

In another aspect, there is provided a method for enhancing the therapeutic outcome in a patient suffering from a cancer expressing PD-L1, which patient is to be treated with nanoparticle complexes comprising albumin, paclitaxel and anti-PD-L1 antibodies, which method comprises treating the patient with a sub-therapeutic amount of the anti-PD-L1 antibody within +/−0.5 hours of administration of the nanoparticles.

In another aspect is provided a method for enhancing the therapeutic outcome in a patient suffering from a cancer overexpressing PD-L1 which patient has been treated with a sub-therapeutic amount of the anti-PD-L1 antibody, the method comprising treating the patients with an effective amount of nanoparticles comprising albumin-bound paclitaxel and anti-PD-L1 antibodies within +/−0.5 hours of administration of the antibodies.

In some embodiments the anti-PD-L1 antibody is administered prior to the carrier protein (e.g., albumin)/paclitaxel/anti-PD-L1 antibody complex, for example, the anti-PD-L1 antibody can be administered minutes, hours or days prior to administration of the carrier protein (e.g., albumin)/paclitaxel/anti-PD-L1 antibody complex. In some embodiments, the anti-PD-L1 antibody is administered between about 5 to about 59 minutes, about 10 to about 50 minutes, about 15 to about 45 minutes, about 20 to about 40 minutes, about 25 to about 35 minutes prior to administration of the carrier protein (e.g., albumin)/paclitaxel/anti-PD-L1 antibody nanoparticle complex. In other embodiments, the anti-PD-L1 antibody can be administered about 1 hour, about 2 hours, about 3 hours, about 4 hours, about 5 hours, about 6 hours, about 12 hours, about 24 hours, about 48 hours, about 72 hours, or longer prior to administration of the carrier protein (e.g., albumin)/paclitaxel/anti-PD-L1 antibody complex. In other embodiments, the anti-PD-L1 antibody can be administered about 1 day, about 2 days, about 3 days, about 4 days, about 5 days, about 6 days, about 7 days, about 10 days, about 12 days, about 15 days, or longer prior to administration of the carrier protein (e.g., albumin)/paclitaxel/anti-PD-L1 antibody complex. Contemplated values include any value, subrange, or range within any of the recited ranges or values, including endpoints.

In some embodiments, the anti-PD-L1 antibody can be administered concurrently with administration of the carrier protein (e.g., albumin)/paclitaxel/anti-PD-L1 antibody complex, for example, within 10 minutes or less of each other.

In other embodiments, the anti-PD-L1 antibody can be administered subsequent to administration of the albumin/paclitaxel/anti-PD-L1 antibody complex, for example, within 2 hours after administration of the albumin/paclitaxel/anti-PD-L1 antibody complex.

Cancers or tumors that can be treated by the compositions and methods described herein include, but are not limited to: biliary tract cancer; brain cancer, including glioblastomas and medulloblastomas; breast cancer; cervical cancer; choriocarcinoma; colon cancer; endometrial cancer; esophageal cancer, gastric cancer; hematological neoplasms, including acute lymphocytic and myelogenous leukemia; multiple myeloma; AIDS associated leukemias and adult T-cell leukemia lymphoma; intraepithelial neoplasms, including Bowen's disease and Paget's disease; liver cancer (hepatocarcinoma); lung cancer; lymphomas, including Hodgkin's disease and lymphocytic lymphomas; neuroblastomas; oral cancer, including squamous cell carcinoma; ovarian cancer, including those arising from epithelial cells, stromal cells, germ cells and mesenchymal cells; pancreas cancer; prostate cancer; rectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma[teratomas, choriocarcinomas]), stromal tumors and germ cell tumors; thyroid cancer, including thyroid adenocarcinoma and medullar carcinoma; and renal cancer including adenocarcinoma and Wilms tumor. Cancers suitable for treatment with the methods described herein include but are not limited to cancers that express or overexpress PD-L1. Cancers that may be treated with the methods disclosed herein also include cancers that are resistant to treatment with anti-PD-L1 antibody immunotherapy, including cancers that had been responsive to immunotherapy but developed resistance to the anti-PD-L1 antibody immunotherapy, which anti-PD-L1 antibodies are not in complex with a nanoparticle comprising a carrier protein (e.g., albumin)-bound chemotherapeutic (e.g., ABRAXANE®).

### Antibody Formulations

In one aspect, the anti-PD-L1 antibody is a unit-dose formulation of an anti-PD-L1 antibody which formulation comprises from about 1% to about 60% of a therapeutic dose of the antibody, wherein the formulation is packaged so as to be administered as a unit dose. In an aspect of the invention, the unit-dose formulation of an anti-PD-L1 antibody comprises about 10% of a therapeutic dose of the antibody. For example 10% of a therapeutic dose of an anti-PD-L1 antibody, e.g., atezolizumab, may be 60 mg to 240 mg.

The unit-dose formulation of an anti-PD-L1 antibody can be about 1% to about 60%, about 5% to about 50%, about 10% to about 40%, about 15% to about 30%, about 20% to about 25%, of a therapeutic dose of the anti-PD-L1 antibody. Contemplated values include any value, subrange, or range within any of the recited ranges, including endpoints.

In some embodiments, the anti-PD-L1 antibody is atezolizumab or a biosimilar version thereof, which formulation comprises from about 5% to about 20% of a therapeutic dose of atezolizumab or a biosimilar version thereof.

In another aspect, provided herein is a formulation comprising an anti-PD-L1 antibody provided herein, and at least one pharmaceutically acceptable excipient.

In general, the unit-dose formulations provided herein can be formulated for administration to a patient by any of the accepted modes of administration. Various formulations and drug delivery systems are available in the art. See, e.g., Remington's Pharmaceutical Sciences, edited by E. W. Martin (Mack Publishing Company, 18th ed., 1990).

In general, unit-dose formulations provided herein will be administered as pharmaceutical compositions by any one of the following routes: oral, systemic (e.g., transdermal, intranasal or by suppository), or parenteral (e.g., intramuscular, intravenous or subcutaneous) administration.

The unit-dose formulations may be comprised of, in general, an anti-PD-L1 antibody, optionally in combination with at least one pharmaceutically acceptable excipient. Acceptable excipients are non-toxic, aid administration, and do not adversely affect the therapeutic benefit of the claimed compounds. Such excipient may be any solid, liquid, semisolid or, in the case of an aerosol composition, gaseous excipient that is generally available to one of skill in the art.

### Nanoparticle Complex Formulations

In one aspect, the composition comprising the nanoparticle complexes described herein is formulated for systemic delivery, e.g., intravenous administration.

In one aspect, the nanoparticle composition is formulated for direct injection into a tumor. Direct injection includes injection into or proximal to a tumor site, perfusion into a tumor, and the like. Because the nanoparticle composition is not administered systemically, a nanoparticle composition is formulated for direct injection into a tumor may comprise any average particle size. Without being bound by theory, it is believed that larger particles (e.g., greater than 500 nm, greater than 1 μm, and the like) are more likely to be immobilized within the tumor, thereby providing what is believed to be a better beneficial effect.

In another aspect, provided herein is a composition comprising a compound provided herein, and at least one pharmaceutically acceptable excipient.

In general, the compounds provided herein can be formulated for administration to a patient by any of the accepted modes of administration. Various formulations and drug delivery systems are available in the art. See, e.g., Gennaro, A. R., ed. (1995) Remington's Pharmaceutical Sciences, 18th ed., Mack Publishing Co.

In general, compounds provided herein will be administered as pharmaceutical compositions by any one of the following routes: oral, systemic (e.g., transdermal, intranasal or by suppository), or parenteral (e.g., intramuscular, intravenous or subcutaneous) administration.

The formulations described herein may include excipients. Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols. Other suitable pharmaceutical excipients and their formulations are described in Remington's Pharmaceutical Sciences, edited by E. W. Martin (Mack Publishing Company, 18th ed., 1990).

The present formulations may, if desired, be presented in a pack or dispenser device containing a unit-dose of the active ingredient. Such a pack or device may, for example, comprise metal or plastic foil, such as a blister pack, or glass, and rubber stoppers such as in vials. The pack or dispenser device may be accompanied by instructions for administration. Compositions comprising a unit-dose formulation of the invention formulated in a compatible pharmaceutical carrier may also be prepared, placed in an appropriate container, and labeled for treatment of an indicated condition.

### Kits

In some aspects, the current invention relates to kits comprising: (a) an amount of albumin-/paclitaxel/anti-PD-L1 antibody nanoparticle complexes, (b) a unit dose of a sub-therapeutic amount of anti-PD-L1 antibody, and optionally (c) instructions for use.

In some embodiments, the kits can include lyophilized complexes of the albumin/paclitaxel/anti-PD-L1 antibody.

In some preferred embodiments, the kit components can be configured in such a way that the components are accessed in their order of use. For example, in some aspects the kit can be configured such that upon opening or being accessed by a user, the first component available is the unit dose of a sub-therapeutic amount of anti-PD-L1 antibody, for example, in a first vial. A second container (e.g., a vial) comprising or containing an amount of the albumin/paclitaxel/anti-PD-L1 antibody nanoparticle complexes can then be accessed. As such the kits can be intuitively configured in a way such that the first vial must be opened prior to the second vial being opened. It should be understood that in some embodiments, the order can be different, for example, where it is desired to administer the complex first, prior to the administration of the antibody. Also, it can be configured such that both are administered at the same time. Finally, it should be understood that additional vials or containers of either or both component(s) can be included, and configured for opening in any desired order. For example, the first vial could be antibody, the second vial could include complex, a third could include either antibody or complex, etc. It is contemplated that a kit configured in such a way would prevent, or at least help to prevent, the components from being administered in an order not intended by the instructions for use.

In some aspects, the invention is directed to a kit of parts for administration of albumin/paclitaxel/anti-PD-L1 antibody complexes and a unit dose of a sub-therapeutic amount of anti-PD-L1 antibody; and optionally further comprising a dosing treatment schedule in a readable medium. In some embodiments, the dosing schedule includes the sub-therapeutic amount of anti-PD-L1 antibody required to achieve a desired average serum level is provided. In some embodiments, the kit of parts includes a dosing schedule that provides an attending clinician the ability to select a dosing regimen of the sub-therapeutic amount of anti-PD-L1 antibody based on the sex of the patient, mass of the patient, and the serum level that the clinician desires to achieve. In some embodiments, the dosing treatment is based on the level of circulating PD-L1 in the blood of the patient. In some embodiments, the dosing schedule further provides information corresponding to the volume of blood in a patient based upon weight (or mass) and sex of the patient. In an embodiment, the storage medium can include an accompanying pamphlet or similar written information that accompanies the unit dose form in the kit. In an embodiment, the storage medium can include electronic, optical, or other data storage, such as a non-volatile memory, for example, to store a digitally-encoded machine-readable representation of such information.

The term “readable medium” as used herein refers to a representation of data that can be read, for example, by a human or by a machine. Non-limiting examples of human-readable formats include pamphlets, inserts, or other written forms. Non-limiting examples of machine-readable formats include any mechanism that provides (i.e., stores and/or transmits) information in a form readable by a machine (e.g., a computer, tablet, and/or smartphone). For example, a machine-readable medium includes read-only memory (ROM); random access memory (RAM); magnetic disk storage media; optical storage media; and flash memory devices. In one embodiment, the machine-readable medium is a CD-ROM. In one embodiment, the machine-readable medium is a USB drive. In one embodiment, the machine-readable medium is a Quick Response Code (QR Code) or other matrix barcode.

### EXAMPLES

The present disclosure is illustrated using a pre-treatment of atezolizumab (i.e., TECENTRIQ™) followed by nanoparticles composed of albumin-bound paclitaxel (i.e., ABRAXANE®) and atezolizumab (i.e., TECENTRIQ™).

One skilled in the art would understand that making and using the nanoparticles, as well as administration of a co-treatment of atezolizumab, of the Examples are for the sole purpose of illustration, and that the present disclosure is not limited by this illustration.

Any abbreviation used herein, has normal scientific meaning. All temperatures are ° C. unless otherwise stated. Herein, the following terms have the following meanings unless otherwise defined:

| | |
|---|---|
| ABX = | ABRAXANE ®/(albumin-bound paclitaxel) |
| ATZ = | atezolizumab |
| BSA = | bovine serum albumin |
| kg = | kilogram |
| nM = | nano molar |
| mg | milligram |
| ml or mL = | milliliter |
| m² = | square meters |
| mm³ = | cubic millimeter |
| μg = | microgram |
| μl = | microliter |
| μm = | micrometer/micron |
| PBS = | Phosphate buffered saline |

### Example 1: Making Atezolizumab-ABRAXANE® Nanoparticles

Atezolizumab and ABRAXANE® (ABX) were co-incubated at room temperature for 30 minutes at a concentration of 4 mg/mL and 10 mg/mL, respectively to form the nanoparticle, AA130.

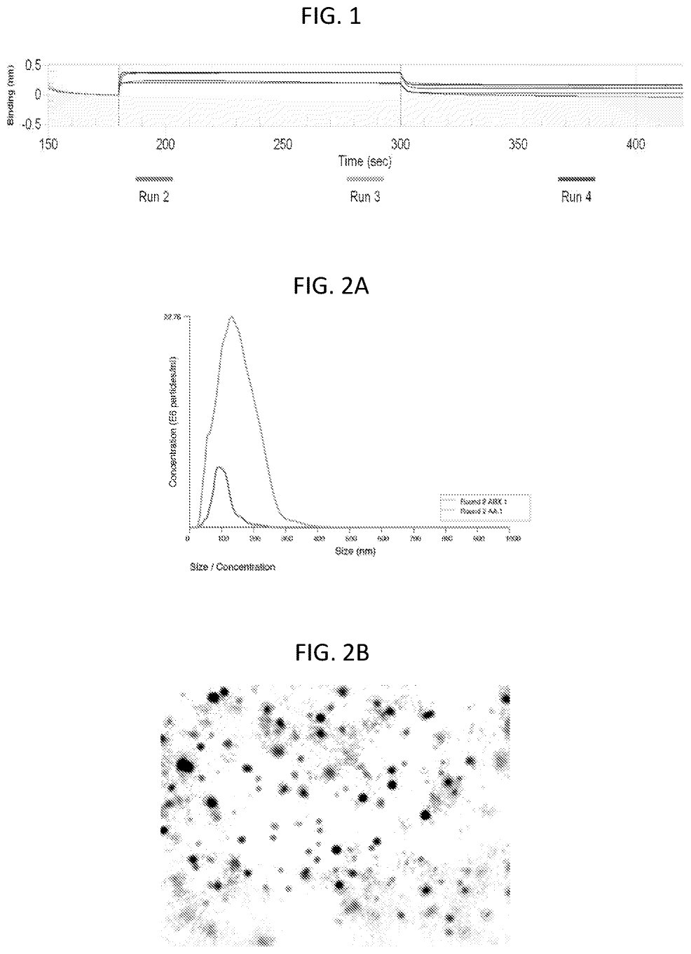

To determine whether atezolizumab and ABX are capable of interacting to form nanoparticle complexes, Biolayer interferometry (BLItz) (Forte Bioscience) was performed using streptavidin probes. 100 ug/ml of biotinylated atezolizumab in 1×PBS was bound to the streptavidin probe. After washing unbound atezolizumab from the probe, the antibody-bound probe was exposed to ABX at concentrations of 100, 500, 1000 μg/mL in 1X PBS. An antibody probe exposed to PBS was used as background and background was subtracted. BLItz software was used to calculate dissociation constants (FIG. 1). The Kd was determined to be 1.462×10⁻⁹.

### Example 2: Size Determination of Atezolizumab-ABRAXANE® Nanoparticles

Mastersizer NS300 was employed to determine the particle size of atezolizumab bound ABX relative to ABX alone. Nanosight uses dynamic light scattering and Brownian motion to calculate particle size.

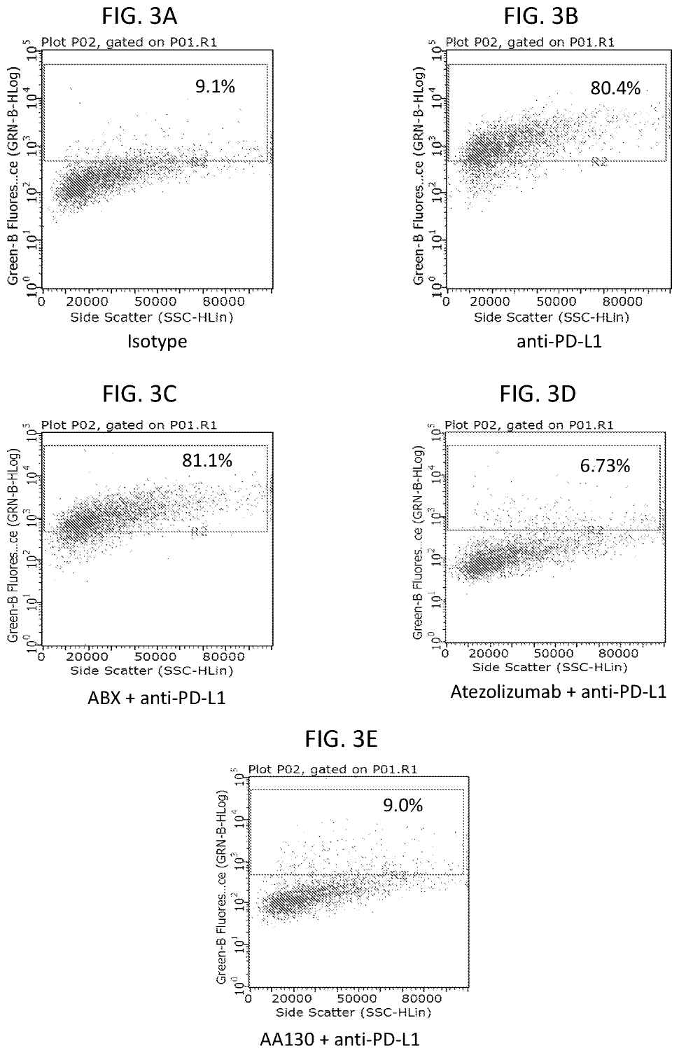

Atezolizumab and ABX were co-incubated to form the nanoparticle, AA130, as described above. ABX was diluted 1:200 and atezolizumab-bound ABX was diluted 1:800; three 30-second video clips were captured and analyzed to determine particle size (FIG. 2A). FIG. 2B is a still image from one of the video clips of AA130. The average particle size of the atezolizumab-ABX nanoparticles was determined to be about 129 nm; average size of ABX alone is about 90 nm.

### Example 3: AA130 Binds PD-L1

Flow cytometry was performed to access binding of atezolizumab and atezolizumab bound Abraxane to the ligand, PD-L1. The PD-L1 positive melanoma cell line, C8161 was used for this experiment. AA130 was made as described above and an aliquot of the nanoparticles was spun at 6000 rpm for 10 minutes to remove any unbound atezolizumab. C8161 cells were stained with FITC labeled isotype control and anti-human PD-L1 as negative and positive controls, respectively. The C8161 cells were incubated for 30 minutes with ABX and atezolizumab alone and the AA130 nanoparticle. After the incubation the cells were labeled with FITC labeled anti-human PD-L1 for 30 minutes and washed with FACS buffer (1×PBS+0.5% BSA and 0.05% Na azide). After washing, the cells were analyzed by flow cytometer on the Guava 8HT and data analysis performed with Gauvasoft software (Millipore).

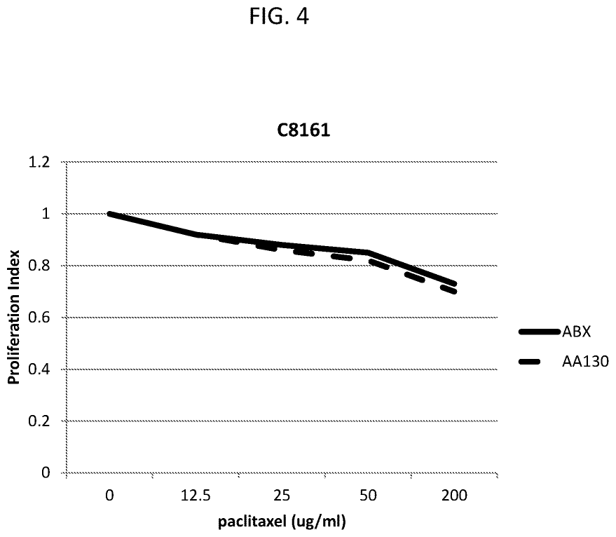

C8161 cells were pre-treated with isotype control antibody (FIG. 3A), no treatment (FIG. 3B), ABRAXANE® (FIG. 3C), atezolizumab (FIG. 3D), or AA130 (FIG. 3E), then labeled with fluorescently-labeled anti-PD-L1 antibody. The atezolizumab in the context of the 130 nm particle retains its ability to bind its ligand, PD-L1.

### Example 4: AA130 Cellular Toxicity

C8161 melanoma cells were exposed to ABX and AA130 at paclitaxel concentrations from 0 to 200 μg/mL overnight to determine cell toxicity. The cells were also incubated with EdU, a thymidine analog. The next day the cells were harvested, fixed with 2% paraformaldehyde and permeabolized with 1% saponin. After permeabolization the cells were incubated for 30 minutes with a FITC labeled anti-EdU antibody to determine the percentage of cells proliferating. After washing, the cells were analyzed by flow cytometer on the Guava 8HT and data analysis performed with Gauvasoft software (Millipore). The proliferation index was calculated by normalization to an untreated positive control.

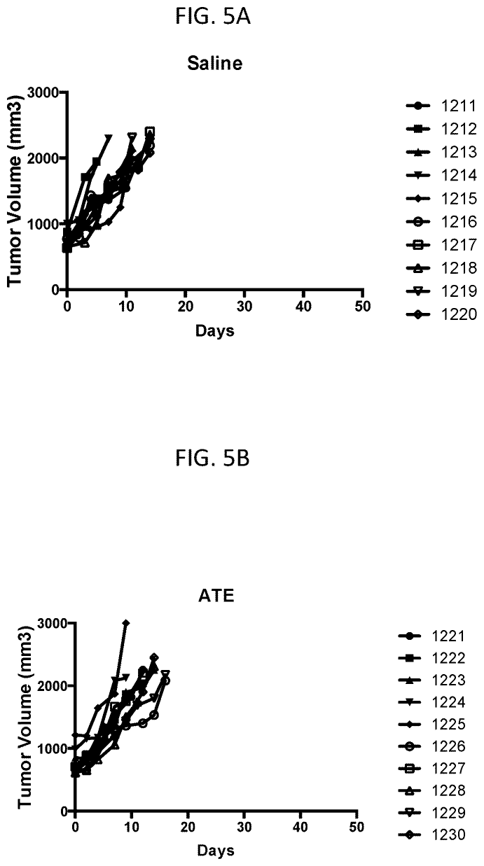

FIG. 4 shows the dose-dependent toxicity of ABX (solid line) and AA130 (broken line) on C8161 cells. The AA130 nanoparticle complex has cellular toxicity similar to ABX alone.

### Example 5: Making of Intravenous Formulation of AA130 Nanoparticle Complexes

For use in humans, the AA130 complexes are prepared by obtaining the dose appropriate number of 4 mL vials of 25 mg/mL ATZ and diluting each vial per the following directions to 4 mg/mL. The dose appropriate number of 100 mg vials of ABX is prepared by reconstituting to a final concentration containing 10 mg/mL ABX nanoparticles. Using a sterile 3 mL syringe, 1.6 mL (40 mg) of atezolizumab (25 mg/mL) is withdrawn and slowly injected, over a minimum of 1 minute, onto the inside wall of each of the vials containing 100 mg of ABX. The atezolizumab solution should not be injected directly onto the lyophilized cake as this will result in foaming. Then, using a sterile 12 mL sterile syringe, 8.4 mL 0.9% Sodium Chloride Injection, USP, is withdrawn and slowly injected, over a minimum of 1 minute, 8.4 mL onto the inside wall of each vial containing ABX 100 mg and ATZ 40 mg. Once the addition of ATZ 1.6 mL and 0.9% Sodium Chloride Injection, USP 8.4 mL is completed, each vial is gently swirled and/or inverted slowly for at least 2 minutes until complete dissolution of any cake/powder occurs. Generation of foam should be avoided. At this point, the concentration of each vial should be 100 mg/10 mL ABX and 40 mg/10 mL ATZ. The vials containing the ABX and ATZ should sit for 60 minutes. The vial(s) is gently swirled and/or inverted every 10 minutes to continue to mix the complex. After 60 minutes has elapsed, the calculated dosing volume of ABX and ATZ is withdrawn from each vial and is slowly added to an empty viaflex bag. An equal volume of 0.9% Sodium Chloride Injection, USP is then added to make the final concentration of ABX 5 mg/mL and ATZ 2 mg/mL. The bag is then be gently swirled and/or inverted slowly for 1 minute to mix. The ABX:ATZ nanoparticles are stored for up to 4 hours at room temperature following final dilution.

### Example 6: Co-Treatment with ATZ Improves Targeting of ABX/ATZ Complexes

Athymic nude mice are injected with 1×10⁶ A375 human melanoma cells in the right flank and then are treated with PBS, 12 mg/kg ATZ, 30 mg/kg ABX, AA130, or pretreated with 1.2 mg/kg ATZ and, 24 hr later, AA130. AA130 are prepared as described in PCT Application No. PCT/US15/54295 and Example 1 above. It is contemplated that only mice treated with AA130 (with or without pretreatment with ATZ) will show reduction in average tumor volume.

It is also contemplated that pretreatment with ATZ will be associated with a statistically significant reduction in tumor volume over control or ATZ alone, or ABX alone.

Tumors are measured on day 15 following treatment with either saline (PBS), TECENTRIQ™ (ATZ), ABRAXANE® (ABX), AA130, or a pretreatment of ATZ one day before AA130 (ATZ+AA130). A 10% sub-therapeutic dose of ATZ, as compared to the dose give to the ATZ alone or AA130 cohort, is given to the ATZ+AA130 cohort 24 hours prior to administration of the AA130. It is contemplated that the ATZ+AA130 cohort will present with delayed tumor growth, even when compared to AA130. It is contemplated that these experiments will show that pre-treatment with ATZ+AA130, increases survival.

Survival is again assessed at day 40. It is contemplated that median survival of mice treated with ATZ pretreatment and AA130 will exceed median survival or the mice treated with either PBS or ATZ alone.

### Example 7: Fluorescence Over Time of AlexaFluor 750 Labeled Nanoparticles

Mice are injected IV with equal amounts of either labeled ABRAXANE®, or nanoparticles of ABRAXANE® having surface complexation with atezolizumab (ATZ) as per Example 1 above (AA130); one AA130 group of mice receives a pre-treatment of 1.2 mg/kg atezolizumab. Fluorescent imagery is performed at an excitation/emission spectrum of 710/760. Regions of interest (ROI) in the mice are assigned by software to track tumor accumulation based on a fluorescence threshold. Fluorescence per unit area of background ROIs and tumor ROIs for all three treatment groups is determined at 24, 29, and 48 hours post injection

The amount of fluorescence (and thus paclitaxel) in the tumor and background ROIs at 24, 29 and 48 hour are determined and it is contemplated that the data will demonstrate that pretreatment with ATZ results in higher levels of tumor fluorescence as compared AA130 alone or ABRAXANE alone. It is contemplated that pretreatment with ATZ and use of ABRAXANE® nanoparticles having surface complexation with ATZ provides for a method for increasing the duration of tumor uptake of albumin containing a chemotherapeutic agent both at 24 hours and 48 hours. It is also contemplated that use of ABRAXANE® nanoparticles having surface complexation with ATZ also provides for increasing the duration of tumor uptake of these albumin containing nanoparticles with or without pretreatment with ATZ at 48 hours.

Without being limited to any theory, the antibody coating of the albumin nanoparticles imparts stability possibly by reducing liver or kidney clearance and/or by reducing protease degradation of the albumin carrier.

### Example 8: In Vivo Efficacy of AA130 Nanoparticles

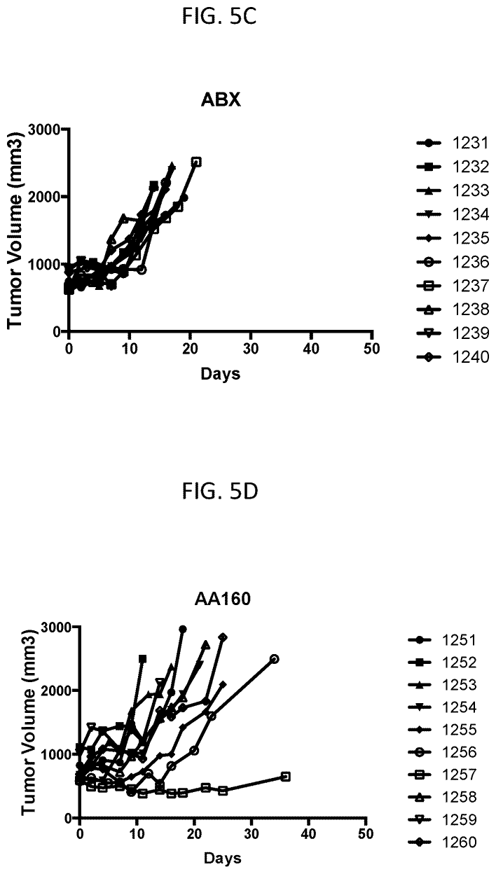

Athymic nude mice (Harlan Sprague Dawley) were injected with 2×10⁶ PD-L1 positive C8161 melanoma tumor cells. The tumors were allowed to grow until about 600 mm³ and were treated by 100 μl IV tail vein injection with saline, atezolizumab alone (18 mg/kg), ABX alone (45 mg/kg) and AA130 (18 mg/kg atezolizumab and 45 mg/kg ABX) one time (FIGS. 5A-5D). Tumor growth was monitored 3 times/week. Tumor size was calculated with the equation: (length×width²)/2.

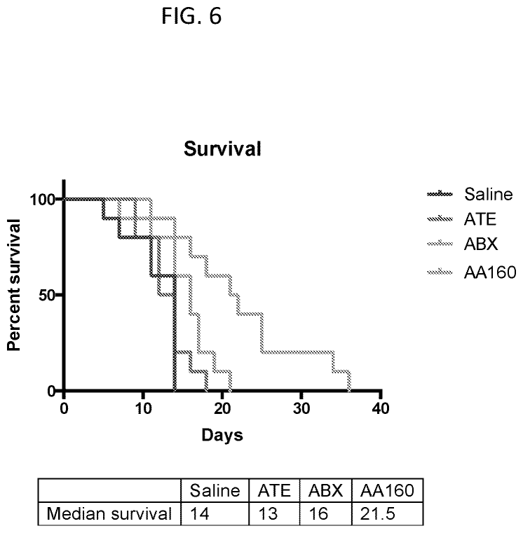

Tumor growth curves (FIG. 6) show slowed tumor growth in the mice treated with AA130 relative to saline and the individual drugs alone. Kaplan Meier curves were generated using Graph Pad software. The median survival for each group was 14, 13, 16, and 21.5 days for saline, atezolizumab, ABX and AA130, respectively. Survival differences between AA130 and all other groups were significant with p-values of 0.0008 for saline, 0.0015 for atezolizumab, and 0.0113 for Abraxane.

1-27. (canceled)

28. A kit comprising: (a) an amount of an albumin/paclitaxel/anti-PD-L1 antibody nanoparticle complexes, (b) a unit dose of a sub-therapeutic amount of anti-PD-L1 antibody, and optionally (c) instructions for use.

29. The kit of claim 28, wherein the albumin/paclitaxel/anti-PD-L1 antibody nanoparticle complexes are lyophilized.

30.-68. (canceled)
